(19) United States
(12) Patent Application Publication
(10) Pub. No.: US 2015/0257752 A1
(43) Pub. Date: Sep. 17, 2015

(54) TISSUE DISSECTION AND LIGATION DEVICE

(71) Applicant: Vitaly Vitalievich Gerbov, Balashiha (RU)

(72) Inventor: Vitaly Vitalievich Gerbov, Balashiha (RU)

(21) Appl. No.: 14/437,212

(22) PCT Filed: Oct. 22, 2012
(86) PCT No.: PCT/RU2012/000850

§ 371 Date: Apr. 21, 2015

(51) Int. Cl. A61B 17/04; A61B 17/12

(52) U.S. Cl. CPC A61B 17/0469; A61B 17/0482; A61B 17/12013; A61B 2017/0474

(57) ABSTRACT

The device generally relates to medicine, more particularly to surgery, and may be used to carry out dissection and occlusion of tubular structures and organs via ligating them with surgical sutures. The invention improves improving the reliability of the application of sutures with a body with control tools at the proximal end and at the distal end, the suture retriever with cutting elements and suture fixators, the device for capturing and clamping the tissue that is provided with oppositely located clamping jaws and knot fixators, and also the body that has pivoted clip with cutting edges for cutting free ends of ligations and transmitting gear, implemented capable of transmitting the controlling action from the control tools to the device for capturing and clamping the tissue, the suture retriever, and the pivoted clip, wherein the device for capturing and clamping the tissue is provided tissue fixation elements located on the clamping jaws.

This application is a National Phase application from PCT application PCT/RU2012/00850 filed on Oct. 22, 2012.

The present invention relates generally to medicine, and more particularly, to surgery and it may be used both for traditional—“open”, and for endosurgical operations to carry out dissection and occlusion of tubular (pipe-like) structures and organs via ligating them with surgical sutures.

During many surgical operations there is a need in transection and occlusion of tissues and organs that comprise tubular structures, such as blood vessels, canals, fallopian tubes, appendix etc. Currently, those manipulations are often carried out manually, and it not only needs the surgeon to have specific skills, but also it needs substantial efforts from the surgeon. That is especially important for endosurgery, where the operative treatment is to be carried out within limited space, with limited amount of access ports, with specific tools that are long, but small in diameter. Besides, despite modified technique used to carry out the occlusion (using Roeder and Melzer loops, clipping with U-shaped metal and plastic clamps, using electrocoagulation), and despite a large number of available accessories now available (knot delivery tools, Roeder loops, unipolar and bipolar electrocoagulators, clamps applicators), there is still a need to frequently replace one tool with another, thus increasing the labor intensity and the duration of the surgical procedure. As I.V. Fedorov noticed, “Any surgery may be schematically divided into three stages: separation of tissues, bleeding control, reconnection of tissues. Each of these stages requires time, specific material and instruments. That makes it natural for a surgeon to try to unite several movements or even stages into one, as well as several instruments into one universal instrument allowing cutting the time taken and the number of the operation stages involved”.

Currently, there are several analogs of the claimed invention known.

One close analog of the proposed technical solution is dual ligation and dividing apparatus [U.S. Pat. No. 5,336,229, A61B17/00, Aug. 19, 1994] with a slot to position a tissue structure, at the distal end of the shaft, with tools to thread two sutures. Herein, each suture is provided with a tip on a distal end, and with a knotted loop on a proximal end, the slot has tools to fix knotted loops. The analog apparatus is provided with moving tools to catch sutures distal ends with the tips to draw the sutures distal ends through the knotted loops to form loops that occlude the tissue. The loops tightened on the tissue are fixed with additional knotted loops. To do so, the analog has tools to tighten the knotted loops. The apparatus also is provided with a cutter to transect the tissue in between the ligations applied, and also with blades to transect free ends of the sutures that are located within the apparatus body.

This device has following disadvantages:
the ligation reliability is relatively low, because the knot that is formed by the apparatus, does not fix the suture tightened on tissues reliably enough. That makes it possible for the suture to loosen, especially when working with dense and elastic structures with big diameter (appendix, fallopian tubes etc.);
usage and maintenance is relatively complicated, due to the presence of multiple working elements and a large number of structural parts.

The technical solution that is the closest to the device proposed, by the technical essence, is the technical solution [RU 2209603, A61B17/12 Aug. 10, 2003] that has a body with distal and proximal ends, control tools at the proximal end, and the device for capturing and clamping the tissue in two points at the distal end of the instrument. The prototype device has a suture retriever that is provided with suture fixators and with cutting elements to transect the tissue. The suture retriever is implemented capable of axial movement between the clamped sites and drawing the sutures beyond the distal end. The outer surface of the device for capturing and clamping the tissue, hosts knot fixators provided with cutting edges to cut off free ends of ligations.

This technical solution has the device for capturing and clamping the tissue in two points implemented as a grabber and a holddown with two distal ends and suture retriever guides in between, while, in another aspect, it was implemented as a combination of two forceps (clamps), each consisting of two hinged jaws. The device body hosts a pivoted clip capable of rotating around the body and having cutting edges to cut free ends of ligation sutures at the distal end.

With the prototype device, capturing and camping of the ligated tissue site is carried out, between the grabber and the holddown, or, depending on the particular implementation, between the forceps jaws, thus fixing the tissue within the device for capturing and clamping the tissue to make its transection possible. Herein, the tissue fixation level is determined by the force of friction, the value of which is determined by the compression effort of the abovementioned elements of the device for capturing and clamping the tissue as well as by the area of contact between the tissue and the working jaws of the device for capturing and clamping the tissue.

When the suture retriever is moved forward, the cutting elements of the suture retriever transect the tissue that is fixed between the clamped sites, with simultaneous running on ligation loops of the Roeder type onto the transected ends of the tissue. Tightening the loops and cutting the free ends of ligations are carried out via means hosted by the apparatus. Herein, the prototype, as well as the claimed device, allows dual-side ligation and transection of the tissue in between the two clamped sites with Roeder ligations. Besides, it has common elements with the invention claimed, because it also has a body with distal and proximal ends, the device for capturing and clamping the tissue in two points with knot fixators and the suture retriever provided with suture fixators and cutting elements.

However, the prototype device has a substantial disadvantage—its reliability is relatively low when used for ligating rather dense, elastic and slick anatomic structures (appendix, fallopian tubes, mainstream vessels etc.) resulting from jamming the suture retriever due to the structure sliding off the device for capturing and clamping the tissue during the transection.

This results from the mechanical features of the prototype, wherein the fixation is carried out via the force of friction that appears after the tissue is pressed within the device for capturing and clamping the tissue, while the tissue transection is carried out strictly in one plane, via the onward, “chopping” movement of the suture retriever directed perpendicular to the surface of the target to cut. Herein, the absent are “sawing” movements to be directed along the surface of the target, which present, for example, during traditional tissue cutting with a scalpel, and which need minimum efforts. The transection principle, as implemented in the prototype, needs substantial efforts to be applied to carry out the transection, and it, if combined with a dense, slick and elastic wall of the target that is ligated (appendix, fallopian tubes and some others), ultimately results in slipping the tissue held off the device for capturing and clamping the tissue drawn by the suture retriever. With the suture retriever moving further on, the dislocated fragments of the tissue get in between the suture retriever and its guides on the device for capturing and clamping the tissue, resulting in jamming the suture retriever and making it impossible to continue using the apparatus to complete the ligation. Besides, if the volume of the tissue causing the jamming is large enough, the prototype can no longer be opened to release the clamped piece of tissue out of the device for capturing and clamping the tissue. Attempts to extract the tissue out of the prototype device are not always efficient and may result in damaging the clamped tissue and developing complications threatening the life of the patient, such as bleeding, or certain aggressive fluids (gut contents, bile etc.) to reach the inner cavities of the organism.

The object of the present invention is to provide reliable fixation of the tissue fragment within the device for capturing and clamping the tissue that would exclude slipping and drawing it in between the suture retriever and the guides, to prevent the apparatus jamming.

The claimed is the Tissue dissection and ligation device that facilitates ligating with surgical sutures and that has a body with proximal and distal ends. The distal end of the body hosts the device for capturing and clamping the tissue that is provided with the clamping jaws located oppositely, in between which the clamping and the fixation of tubular structure, or the tissue comprising thereof, takes place by means of knots fixators provided with cutting edges to cut free ends of ligations. The distal end of the body hosts the suture retriever that is provided with suture fixators and with the cutting elements to transect the tissue clamped. The body hosts a pivoted clip capable of rotating around the body and having slots on the outer side to position free ends of the ligating sutures with cutting edges to cut the free ends of the ligations. The proximal end of the body hosts control tools that interact with the driving mechanisms of the device for capturing and clamping the tissue, of the suture retriever, of the pivoted clip, and with the means for tightening the free ends of ligations.

The required technical result is achieved in such a way, that the clamping jaws of the Tissue dissection and ligation device have fixation elements preventing the structure clamped from slipping.

To do so, the jaws surfaces have roughnesses to increase the force of friction that acts against slipping. Those roughnesses may be created by means of any technical way known, for example, by creating lowered and raised portions, by spattering particles of any material (metal, diamond dust etc.).

In an alternative embodiment the clamping jaws may be provided with spikes or needles that puncture the structure clamped to provide its mechanical fixation within the device for capturing and clamping the tissue. To achieve full closure of the jaws, opposite the spikes or the needles, holes or lunules are placed on jaws to take in the needles or the spice when the jaws are closed.

Yet another technical solution that prevents slipping the tubular structures during cutting is creating such surfaces upon jaws working surfaces that provide axial bending of the structure clamped when pressed within the device for capturing and clamping the tissue. Herein, resulting from changing the direction of the axis of the tissue clamped, the initial slipping force (F_{sl.in.}) that affects the initial spot of the tissue before bending gets decomposed into the force that holds the clamped tissue down to the support (the clamping jaw surface) (F_{holddown}) and the ultimate force of slipping (F_{ult.slp.}) directed perpendicular to the surface of the support and along the axis of the end fragment of the tissue located after the bending.

F_{sl.in.}=F_{holdown}+F_{ult.slp.}

It comes out of the expression, that the ultimate force of slipping (F_{ult.slp.}) affecting the spot of the tissue located after the bending would be less, than initial slipping force (F_{sl.in.}) for the value of the force that holds the clamped tissue down to the clamping jaw surface (F_{holdown}) resulting in preventing the clamped tissue portion from dislocation within the device for capturing and clamping the tissue.

These phenomena are widely used in daily life, for example, if one needs to hold a cord or fishing line by hand, the cord, or the fishing line, is to be bent around finger or hand, or the hand may be repositioned in such a way, that the cord axis would bend at some angle to the pulling force.

The proposed device provides this effect in such a way, that the clamping jaws of the device for capturing and clamping the tissue have surfaces providing axial bending of the tissue fragment clamped.

For this purpose, the clamping jaws are implemented at an acute angle to the guides of the suture retriever with the vertex directed towards the cutting direction. Resulting therefrom, two axial bendings of the clamped structure are formed. Herein, taking the place is the reduction of the force causing the tissue slipping, the more acute is the angle between the jaws surfaces and the suture retriever guides, and the more substantial is the reduction. Another effect resulting from such a configuration is the reduced cutting effort, because additional cutting edges appear and the cutting takes place via guillotine method by means of interaction between the “on-coming” cutting edges on the suture retriever and the clamping jaws.

Certainly, if multiple bendings of the clamped structure axis were formed, the slipping force would be reduced more substantially. This may be achieved by creating V-shape, Π-shape, or U-shape clutches on the clamping surfaces of the jaws with dimensions sufficient to provide axial bendings of the clamped tissue.

The bonus effect resulting from such working jaws surface configurations would include increasing the area of the clamping surface, thus, the force of friction would increase either, to prevent the clamped structure from slipping.

Tissue dissection and ligation device and the illustrations of work thereof are represented in connection with the following drawings that are divided into groups:

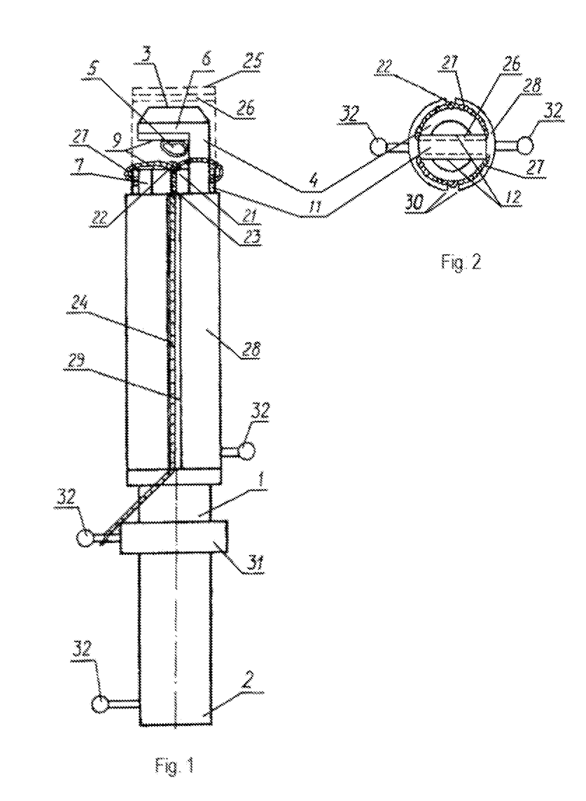

FIG. 1 and FIG. 2 represent the general view of the Tissue dissection and ligation device;

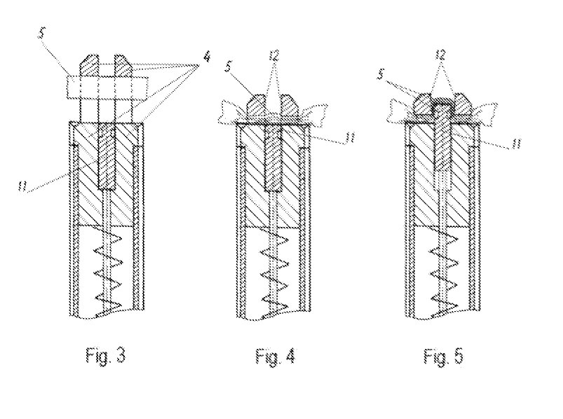

FIG. 3-5 illustrate the mechanism of suture retriever jamming resulting from the tissue slipping from the device for capturing and clamping the tissue and getting the slipped fragment in between the suture retriever and the guides thereof.

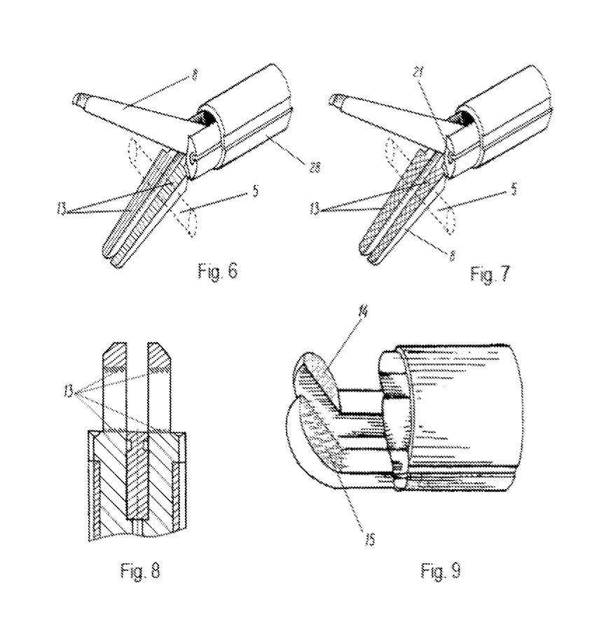

FIG. 6-17 represents embodiments of tissue fixation elements located on the clamping jaws of the device for capturing and clamping the tissue.

Each of the drawings represents:

FIG. 1 represents a Tissue dissection and ligation device with the device for capturing and clamping the tissue in two points implemented as a grabber and a holddown. The lateral view, from the right side (the dotted line represents the full shift position of the suture retriever);

FIG. 2 represents the Tissue dissection and ligation device with the device for capturing and clamping the tissue in two points implemented as a grabber and a holddown. The upper view from the side of the distal end thereof;

FIG. 3 represents the Tissue dissection and ligation device with the device for capturing and clamping the tissue in two points implemented as a grabber and a holddown. The upper view from the side of the distal end thereof, with the frontal plane section. Tissue capturing stage: the tissue is positioned within the device for capturing and clamping the tissue, the device for capturing and clamping the tissue is opened the suture retriever is pushed inside;

FIG. 4 represents the Tissue dissection and ligation device with the suture retriever and with the device for capturing and clamping the tissue in two points, implemented as a grabber and a holddown. The upper view from the side of the distal end thereof, with the frontal plane section. Tissue capturing stage: the tissue is positioned within the device for capturing and clamping the tissue, the device for capturing and clamping the tissue is closed the suture retriever is pushed inside;

FIG. 5 represents the Tissue dissection and ligation device with the suture retriever and with the device for capturing and clamping the tissue in two points implemented as a grabber and a holddown. The upper view from the side of the distal end thereof, with the frontal plane section. Transection stage with the suture retriever jamming developing: due to the suture retriever the tissue slipped off the device for capturing and clamping the tissue to be pulled in between the suture retriever and the guides thereof upon the device for capturing and clamping the tissue. The device for capturing and clamping the tissue is closed, the suture retriever is half pulled forward, any further movement of the suture retriever is complicated or impossible due to the tissue fragment pulled in;

FIG. 6 represents the device for capturing and clamping the tissue in two points implemented as two pairs of hinged jaws. Lateral projection view. The clamping jaws have roughnesses as lowered and raised portions crosswise or lengthwise;

FIG. 7 represents the device for capturing and clamping the tissue in two points implemented as two pairs of hinged jaws. Lateral projection view. The clamping jaws have roughnesses as intercrossing lowered and raised portions located at an angle;

FIG. 8 represents the device for capturing and clamping the tissue in two points, implemented as a grabber and a holddown. The upper view with frontal plane section. The clamping jaws have roughnesses created by spattering particles of metal on jaw surface, diamond dust or any other material;

FIG. 9 represents the device for capturing and clamping the tissue in two points, implemented as a grabber and a holddown. The lateral projection view. The clamping jaws are provided with spikes (right) and needles (left) to fix the tissue mechanically;

FIG. 10 represents the device for capturing and clamping the tissue in two points, implemented as a grabber and a holddown. The upper view with frontal plane section. The clamping jaws implemented at an acute angle to the suture retriever guides with the vertex directed against the cutting direction with possibility to create axial bending of the tissue clamped to form sharp cutting edges. The dotted line marks the tissue axial bending;

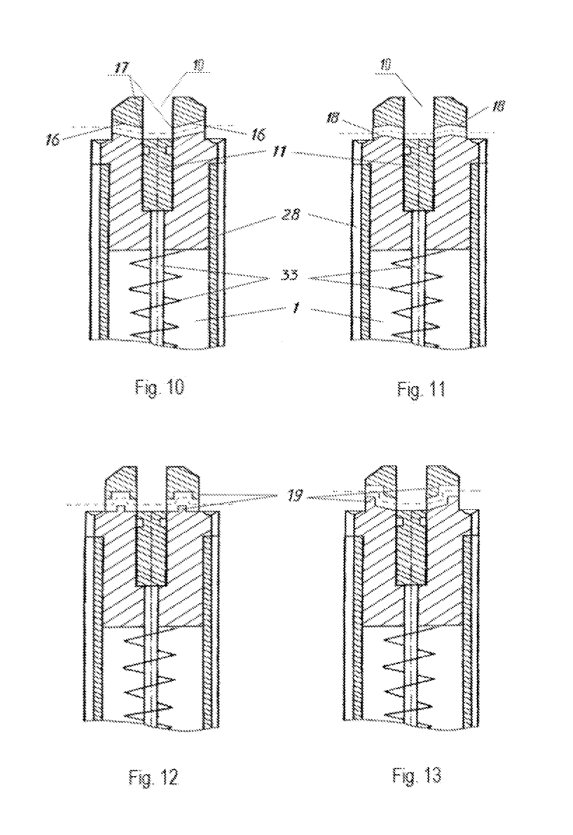

FIG. 11 represents the device for capturing and clamping the tissue in two points, implemented as a grabber and a holddown. The upper view with frontal plane section. The clamping jaws have V-shaped clutches that provide the clamped tissue axial bending. The dotted line marks the tissue axial bending;

FIG. 12 represents the device for capturing and clamping the tissue in two points, implemented as a grabber and a holddown. The upper view with frontal plane section. The clamping jaws have n-shaped clutches that provide the clamped tissue axial bending. The dotted line marks the tissue axial bending;

FIG. 13 represents the device for capturing and clamping the tissue in two points, implemented as a grabber and a holddown. The upper view with frontal plane section. The clamping jaws have the aspect embodiment of the f-shaped clutches that provide the clamped tissue axial bending. The dotted line marks the tissue axial bending;

FIG. 14 represents the device for capturing and clamping the tissue in two points implemented as two pairs of hinged jaws. Lateral view. The clamping jaws have V-shaped clutches that provide the clamped tissue axial bending. The tissue is positioned in the device for capturing and clamping the tissue, the device for capturing and clamping the tissue is opened;

FIG. 15 represents the device for capturing and clamping the tissue in two points implemented as two pairs of hinged jaws. Lateral view. The clamping jaws have V-shaped clutches that provide the clamped tissue axial bending. The tissue is positioned in the device for capturing and clamping the tissue, the device for capturing and clamping the tissue is closed;

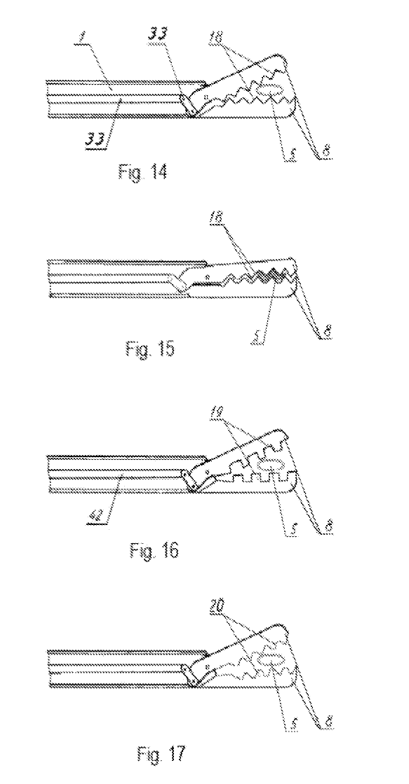

FIG. 16 represents the device for capturing and clamping the tissue in two points implemented as two pairs of hinged jaws. Lateral view. The clamping jaws have Π-shaped clutches that provide the clamped tissue axial bending. The tissue is positioned in the device for capturing and clamping the tissue, the device for capturing and clamping the tissue is opened;

FIG. 17 represents the device for capturing and clamping the tissue in two points implemented as two pairs of hinged jaws. Lateral view. The clamping jaws have U-shaped clutches that provide the clamped tissue axial bending. The tissue is positioned in the device for capturing and clamping the tissue, the device for capturing and clamping the tissue is opened.

The Tissue dissection and ligation device has a body 1 with proximal (the one that is closer to the operator) 2 and the distal (the one that is farther from the operator) 3 ends.

The distal end 3 of the body 1 hosts the device for capturing and clamping the tissue in two points 4, with the tissue portion 5 that is subject for transection and ligation positioned within.

The device for capturing and clamping the tissue in two points 4 is implemented as the tissue grabber 6 and the holddown 7, or as two pairs of co-axially located jaws 8. On the holddown, on the grabber or, alternatively, on the jaws, oppositely (one opposite another), clamping jaws 9 are located wherein the tissue 5 is to be clamped and fixed in between upon the closure thereof.

Located upon the medium line of the device for capturing and clamping the tissue is the slot 10 of the suture retriever 11. Lateral walls of the slot 10 work as guides 12 for the suture retriever 11.

The clamping jaws 9 have roughnesses 13 to improve fixation reliability, that is achieved by means of creating small lowered and raised portions (notches, ridges or by means of spattering small particles of metal or another material etc.) upon the surface of the jaws 9 by means of any technique known in the art. In this aspect, the tissue fixation reliability is achieved via increasing the friction force.

In an alternative embodiment, the clamping jaws are provided with spikes 14 or needles 15 that puncture the clamped tissue 5 to mechanically fix it and to prevent its dislocation and slipping from the device for capturing and clamping the tissue 4.

In yet another embodiment, the clamping jaws 9 are implemented at an acute angle 16 to the suture retriever guides, with the vertex directed towards the cutting direction. Resulting therefrom, the clamped tissue axial bending is created within the device for capturing and clamping the tissue 4 to reduce the force initiating the slipping of the tissue. Another effect resulting from such a configuration is the reduction of the cutting effort due to the creation of sharp cutting edges 17 in between the guides 11 of the suture retriever 12 and the clamping jaws 9 directed towards the cutting effort direction.

To create multiple axial bendings for more substantial reduction of the slipping force, the clamping jaws have V-shaped 18, Π-shaped 19 or U-shaped 20 clutches with the size that is sufficient to provide axial bending of the clamped tissue. Besides, the proposed solution also results in increasing of the clamping surface area, thus further increasing the friction force to prevent slipping the clamped structure from the device for capturing and clamping the tissue.

The outer sides of the device for capturing and clamping the tissue host knot fixators 21 to place ligation knots 22 provided with cutting edges 23 to cut free ends of ligations 24.

The distal end 3 of the device hosts the suture retriever 12 that moves between the guides 11 on the device for capturing and clamping the tissue 4. The suture retriever 12 is provided with the cutting elements 25 to transect the tissue 5 and with the suture fixators 26 with ligation loops 27 within.

The body 1 hosts a pivoted clip 28 that has slots 29 onto the outer side thereof to position the free ends of the ligations 24, and the cutting edges 30 to cut the free ends of ligations 24.

The proximal end 2 of the body 1 hosts a movable hub 31 capable of fixing and tightening the free ends of ligations 24.

The proximal end of the body hosts control tools 32 acting, via transmission gear 33, onto the device for capturing and clamping the tissue 4, the suture retriever 12, the pivoted clip 28, and the movable hub 31.

The proposed device is used as follows.

Preliminarily prepared Roeder loops (or similar thereof) are installed within the device to be located within the suture fixators 26 on the suture retriever 12 and within the knot fixators 21 of the device for capturing and clamping the tissue 4. The free ends of the Roeder loops are fixed onto the movable hub 31. After this, the device is delivered to the portion of the tissue to be ligated that is put in between the clamping jaws. Acting on the control tools, the tissue fragment is fixed between the clamping jaws in the device for capturing and clamping the tissue.

Herein, the uneven surfaces of the working jaws, upon closure thereof, substantially increase the force of friction working against the slipping force, thus excluding the dislocation of the tissue portion that is fixed in the device for capturing and clamping the tissue portion, that otherwise would have resulted in jamming and getting it in between the suture retriever and the guides thereof.

With spikes or needles on the surfaces of the clamping jaws, multiple punctures take place throughout the entire volume of the clamped portion of tissue, thus providing mechanical fixation thereof within the device for capturing and clamping the tissue to prevent slipping.

If the clamping jaws are implemented with an acute angle to the suture retriever guides and with the vertex directed against the cutting direction, axial bendings of the clamped structure are established in the device for capturing and clamping the tissue. This results in reduction of the slipping force due to splitting its vector into components thereof. Besides, in this aspect the cutting effort is reduced either, due to creation of the sharp cutting edges in between the suture retriever guides and the clamping jaws, and this also results in reduction of the force needed for cutting, and, thus, the force that works for slipping, too.

Creation of multiple bendings of the clamped tissue fragment is obtained by means of implementing the clamping jaws as V-shaped, fl-shaped or U-shaped clutches with the size that is sufficient to provide axial bending of the clamped tissue. This results in more substantial reduction of the slipping force acting onto the tissue from the side of the suture retriever, and, besides, in increasing of the clamping surface area, thus further increasing the friction force to prevent slipping the clamped structure from the device for capturing and clamping the tissue.

Thus, all the above mentioned solutions improve the clamped tissue fixation within the device for capturing and clamping the tissue and prevent its dislocation and getting it in between the suture retriever and the guides thereof, thus preventing suture retriever jamming.

With the unrestricted, jam-free ongoing extraction of the suture retriever forward the fixed tissue is transected between the clamped portions. Herein, the ligation loops are brought by the suture retriever beyond the distal end of the apparatus, and they are freed from the suture fixators embracing the transected ends from both sides. Moving the movable hub with the ligation free ends, affixed onto toward the proximal end, and facilitates tightening the ligation loops on the tissue. After that, rotating the pivoted clip, cutting edges interaction cuts the free ends of the ligations. By means of the control tools, the device for capturing and clamping the tissue is opened to move the claimed invention off the ligation place.

1. A tissue dissection and ligation device, comprising: a body with distal and proximal ends, wherein the proximal end of the body is provided with control tools, movable hub, implement capable of affixing free ends of ligations onto, while the distal end is provided with a suture retriever implement capable of axial movement between clamped portions of tissue and of bringing ligations beyond the distal end, the suture retriever has cutting elements and suture fixators and device for capturing and clamping the tissue that has oppositely located clamping jaws, knot fixators implemented capable of feeding the loop knots to the ligation tightening places, and the body is provided with pivoted clip that has cutting edges to cut free ends of ligations, a transmission gear, implement capable of transmitting the controlling action from the control tools onto the device for capturing and clamping the tissue, the suture retriever and the pivoted clip, wherein the device for capturing and clamping the tissue is implemented with elements for tissue fixation on the clamping jaws.

2. The device of the claim 1, wherein the elements for tissue fixation on the clamping jaws are formed by roughness in a form of lowered and raised portions.

3. The device of the claim 1, wherein the elements for tissue fixation on the clamping jaws are formed by roughness in a form of spattering particles of metal, diamond, or another material.

4. The device of the claim 1, wherein the elements for tissue fixation on the clamping jaws are formed by needles, and the corresponding lunules are placed oppositely to take in the the needles when the device for capturing and clamping the tissue is closed.

5. The device of the claim 1, wherein the elements for tissue fixation on the clamping jaws are created by the clamping jaws surfaces implemented at an acute angle to the guides of the suture retriever with the vertex directed against the direction of cutting, capable of creating axial bending of the clamped tissue and with creation of the on-coming cutting edges.

6. The device of the claim 1, wherein the elements for tissue fixation on the clamping jaws are formed by V-shape clutches that facilitate creation of axial bending of the clamped tissue when clamping it with the device for capturing and clamping the tissue.

7. The device of the claim 1, wherein the elements for tissue fixation on the clamping jaws are formed by spikes and corresponding lunules are placed oppositely to take in the spikes when the device for capturing and clamping the tissue is closed.

8. The device of the claim 1, wherein the elements for tissue fixation on the clamping jaws are formed by Π-shape clutches that facilitate creation of axial bending of the clamped tissue when clamping it with the device for capturing and clamping the tissue.

9. The device of the claim 1, wherein the elements for tissue fixation on the clamping jaws are formed by U-shape clutches that facilitate creation of axial bending of the clamped tissue when clamping it with the device for capturing and clamping the tissue.

10. A tissue dissection and ligation device, comprising: a body with distal and proximal ends, wherein the proximal end of the body is provided with control tools, movable hub, implement capable of affixing free ends of ligations onto, while the distal end is provided with a suture retriever implement capable of axial movement between clamped portions of tissue and of bringing ligations beyond the distal end, the suture retriever has cutting elements and suture fixators and device for capturing and clamping the tissue that has oppositely located clamping jaws, knot fixators implemented capable of feeding the loop knots to the ligation tightening places, and the body is provided with pivoted clip that has cutting edges to cut free ends of ligations, a transmission gear, implement capable of transmitting the controlling action from the control tools onto the device for capturing and clamping the tissue, the suture retriever and the pivoted clip, wherein the device for capturing and clamping the tissue is implemented with elements for tissue fixation on the clamping jaws, and wherein the elements for tissue fixation on the clamping jaws are formed by roughness in a form of spattering particles of metal, diamond, or another material.

11. The device of the claim 10, wherein the elements for tissue fixation on the clamping jaws are formed by roughness in a form of lowered and raised portions.

12. The device of the claim 10, wherein the elements for tissue fixation on the clamping jaws are formed by needles, and corresponding lunules are placed oppositely to take in the needles when the device for capturing and clamping the tissue is closed.

13. The device of the claim 10, wherein the elements for tissue fixation on the clamping jaws are created by the clamping jaws surfaces implemented at an acute angle to the guides of the suture retriever with the vertex directed against the direction of cutting, capable of creating axial bending of the clamped tissue and with creation of the on-coming cutting edges.

14. The device of the claim 10, wherein the elements for tissue fixation on the clamping jaws are formed by V-shape clutches that facilitate creation of axial bending of the clamped tissue when clamping it with the device for capturing and clamping the tissue.

15. The device of the claim 10, wherein the elements for tissue fixation on the clamping jaws are formed by spikes and corresponding lunules are placed oppositely to take in the spikes when the device for capturing and clamping the tissue is closed.

16. The device of the claim 10, wherein the elements for tissue fixation on the clamping jaws are formed by n-shape clutches that facilitate creation of axial bending of the clamped tissue when clamping it with the device for capturing and clamping the tissue.

17. The device of the claim 10, wherein the elements for tissue fixation on the clamping jaws are formed by U-shape clutches that facilitate creation of axial bending of the clamped tissue when clamping it with the device for capturing and clamping the tissue.
